(19) United States
(12) Patent Application Publication
(10) Pub. No.: US 2004/0220125 A1
(43) Pub. Date: Nov. 4, 2004

(54) Biosynthetic platform for cardioprotective gene expression using immature heart tissue

(76) Inventors: John G. Coles, Toronto (CA); Glen Van Arsdell, Toronto (CA); Mark Takahashi, Toronto (CA)

Correspondence Address: Michael A. Slavin, Esq., McHale & Slavin, P.A., Suite 402, 4440 PGA Boulevard, Palm Beach Gardens, FL, 33410, US

(21) Appl. No.: 10/429,656

(22) Filed: May 2, 2003

(51) Int. Cl. C12Q 1/68; A61K 48/00; C07H 21/04

(52) U.S. Cl. 514/44; 435/6; 536/23.2

(57) ABSTRACT

The present invention is directed toward identification of cardioprotective gene programs in the neonatal heart. Specifically, the newborn (immature) heart or alternatively fetal heart has been recognized as having an increased resistance to pathophysiological forms of stress, e.g. hypoxic stress. The pattern of gene expression in immature heart subject to naturally occurring hemodynamic and hypoxic stress, e.g. that associated with obstructive congenital heart disease, is herein revealed by differential gene profiling; and the induction of a cardioprotective gene pattern, and particularly useful subsets thereof, in the heart chronically adapted to stress is confirmed. Thus, the chronically stressed immature heart provides a novel biosynthetic platform for cardioprotective gene expression, useful as a basis for the development of diagnostic and therapeutic modalities.

### FIELD OF THE INVENTION

This invention relates to age-specific differential gene expression profiling as a result of naturally occurring disease states; particularly to the determination of a unique cardioprotective gene expression profile by exploitation of the ability of neonatal or fetal cardiac tissue to respond to a complex pathophysiological stress.

### BACKGROUND OF THE INVENTION

The global myocardial stress response during cardiac surgery has not been systematically studied or previously reported. Nor is it known whether there are age related differences in the stress response of the newborn heart in response to the stress of congenital heart surgery, or whether the response of neonatal myocardium is intrinsically different from that of older children.

This global gene expression profile of the heart in response to cardiac surgery has not previously been reported. The instant inventors hypothesized that the neonatal heart has an enhanced stress response due to a greater repertoire of inducible gene activation programs; and that the molecular basis of this response can be revealed by gene expression analysis of the immature (neonatal/fetal) heart which is pathologically stressed by congenital heart disease (CHD).

Neonatal myocardium has been found to exhibit a unique pattern of gene expression. This reflects a stress-induced protective program which includes both novel and precedented genes, which potentially represent important therapeutic targets.

The instant inventors studied gene expression profiles in 24 patients during surgery for tetralogy of Fallot, stratified into a first group (7 pts, aged from 5 to 66 days, mean 30 days) and a second group (17 pts, aged from 4 months to 180 months, mean 33.5 months). Biopsies from the right ventricular outflow tract were taken following aortic occlusion and archived in liquid nitrogen. RNA isolation, fluorescence-labeling of CDNA, hybridization to spotted arrays containing 19,008 characterized or unknown human cDNAs, and quantitative fluoresence scanning of gene expression intensity, were performed at the University of Toronto Health Network Microarray Centre. Data were analyzed with the Significance Analysis for Microarrays program. Minimum Information about Microarray Experiments (MIAME)-compliant, log2-normalized data sets were compared to ascertain potential statistical differences in gene expression between patient groups. Transcripts were identified which were differentially expressed in the neonatal group {predicted false discovery rate <0.8 transcripts}. The dominant pattern of gene expression was consistent with a differential gene expression profile which we term a cardioprotective program, simultaneously exhibiting a combination of evident functional clusters including both up-regulated and down-regulated genes evidencing anti-hypertrophic, anti-fibrotic and pro-vasodilatory programs. As used herein the terms up-regulated and down-regulated are understood to mean that the difference (in this case between immature and mature heart tissue) exceeds a statistical threshold corresponding to a false discovery rate of less than 1 percent as determined using statistical analysis for microarrays (SAM) software. Both the cardioprotective combinatorial pattern of gene expression along with numerous individual transcripts and combinations thereof are unique and have not been previously reported in heart.

Defining this combinatorial pattern, which is viewed by the instant inventors as an “anti-disease network”, and elucidating the components thereof, will provide the clinician with an insight into identifying and determining the molecular basis of the protective genes which account for this enhanced stress response.

Furthermore, the instant inventors propose that relating the herein disclosed anti-disease network to clusters of genes which are abnormally expressed in alternative age groups represents a heretofore unavailable diagnostic tool, useful as an early indicator of asymptomatic cardiac compensation, e.g. in congestive heart failure.

### DESCRIPTION OF THE PRIOR ART

U.S. Pat. No. 6,365,352 relates to a method to identify granulocytic cell genes that are differentially expressed upon exposure to a pathogen or in a sterile inflammatory disease by preparing a gene expression profile of a granulocytic cell population exposed to a pathogen or isolated from a subject having a sterile inflammatory disease and comparing that profile to a profile prepared from quiescent granulocytic cells. The method is particularly useful for identifying cytokine genes, genes encoding cell surface receptors and genes encoding intermediary signaling molecules. The patent also includes methods to identify a therapeutic agent that modulates the expression of at least one gene in a granulocytic population. Genes which are differentially expressed during neutrophil contact with a pathogen, such as a virulent bacteria, or that are differentially expressed in a subject having a sterile inflammatory disease are of particular importance. The referenced patent relates solely to white blood cells and does not reference age-specific gene repertoires.

U.S. Pat. No. 6,461,814 provides a rapid, artifact free, improved method of obtaining short DNA “tag” or arrays thereof, allowing for determination of the relative abundance of a gene transcript within a given mRNA population and is useful to identify patterns of gene transcription, as well as identify new genes. This patent refers to a method of mRNA profiling which is not the subject of the present invention, nor does it infer the novel approach to elucidation of an age-related gene repertoire exploited by the instant invention.

Published Application US20010018182A1 is directed towards methods for monitoring disease states in a subject, as well as methods for monitoring the levels of effect of therapies upon a subject having one or more disease states. The methods involve: (i) measuring abundances of cellular constituents in a cell from a subject so that a diagnostic profile is obtained, (ii) measuring abundances of cellular constituents in a cell of one or more analogous subjects so that perturbation response profiles are obtained which correlate to a particular disease or therapy, and (iii) determining the interpolated perturbation response profile or profiles which best fit the diagnostic profile according to some objective measure. In other aspects, the invention also provides a computer system capable of performing the methods of the invention, data bases comprising perturbation response profiles for one or more diseases and/or therapies, and kits for determining levels of disease states and/or therapeutic effects according to the methods of the invention. The publication utilizes the concept of gene profiling to monitor disease states, however there is no conceptual overlap to the instant invention of obtaining a particular genetic response profile related to an age-related response to disease related stressors.

Published Application US20030008290A1 provides a method for serial analysis of gene expression, SAGE, a method for the rapid quantitative and qualitative analysis of transcripts, has been improved to provide more genetic information about each analyzed transcript. In SAGE, defined sequence tags corresponding to expressed genes are isolated and analyzed. Sequencing of over 1,000 defined tags in a short period of time (e.g., hours) reveals a gene expression pattern characteristic of the function of a cell or tissue. Although SAGE is useful as a gene discovery tool for the identification and isolation of novel sequence tags corresponding to novel transcripts and genes, the reference in no way contemplates the methodology practiced by the instant inventors.

Published Application US20030032030A1 teaches a method of measuring the biological age of a multicellular organism. In one embodiment, the method comprises the steps of: (a) obtaining a sample of nucleic acid isolated from the organism's organ, tissue or cell, wherein the nucleic acid is RNA or a cDNA copy of RNA and (b) determining the gene expression pattern of at least one of the genes selected from the group consisting of M21050, Z49204, U49430, K02782, X58861, X66295, M22531, X67809, U19118, M64086, M63695, U39066, X92590, X56518, AA182189, X16493, U20344, X16834, X82648, D00754, D16313, L38971 and X15789; and Published Application US20030036079A1 is drawn to a method of measuring the relative metabolic state of a multicellular organism is disclosed. In one embodiment, the method comprises the steps of: (a) obtaining a sample of nucleic acid isolated from the organism's organ, tissue or cell, wherein the nucleic acid is RNA or a cDNA copy of RNA, (b) determining the gene expression pattern of at least one of the genes selected from the group consisting of D31966, R74626, U79163, M22531, U43285, U79523, X81059, X84239, D38117, M70642, U37775, U84411, D87117, U31966, U51167, M97900, U32684, U43836, U60001, X61450, D49473, L08651, U28917, U49507, X59846, X00958, K03235, Z48238, M60596, AA117417, AF007267, AF011644, AJ001101, C79471, D16333, D49744, D83146, D86424, L29123, L40632, M74555, M91380, M93428, U19799, U20344, U34973, U35312, U35646, U43512, U47008, U47543, U56773, X06407, X54352, X84037, Y00746, Y07688, Z19581, Z46966, AF003695, AF020772, C76063, C79663, D10715, D12713, D67076, D86344, L10244, L18888, M57966, M58564, U19463, U25844, U27830, U35623, U43892, U51204, U75321, U84207, X52914, X54424, X75926, X99921 and Z47088 and (c) determining whether the gene expression profile of step (b) is more similar to a CR-induced metabolic state or a standard diet metabolic state. Both US 23032030A1 and US 23036079A1 focus on senescence-related targets which can be used to retard aging, based on expression profiling in normal rodents; and do not involve stress response, as in the instant invention.

WO 09910535A1 teaches a method to identify stem cell genes that are differentially expressed in stem cells at various stages of differentiation when compared to undifferentiated stem cells by preparing a gene expression profile of a stem cell population and comparing the profile to a profile prepared from stem cells at different stages of differentiation, thereby identifying cDNA species, and therefore genes, which are expressed. Further disclosed are methods to identify a therapeutic agent that modulates the expression of at least one stem cell gene associated with the differentiation, proliferation and/or survival of stem cells. The disclosure's focus is on stem cell gene expression changes during hematopoiesis, not specifically the stress response; no overlap in targets exist with those of the instant disclosure.

W009958720A1 provides methods for quantifying the relatedness of a first and second gene expression profile and for ordering the relatedness of a plurality of gene expression profiles to a single preselected gene expression profile. The methods are demonstrated to be useful for quantifying the relatedness of environmental conditions upon a cell, such as the relatedness in effects of pharmaceutical agents upon a cell. The methods are also useful in quantifying the relatedness of a preselected environmental condition to a defined genetic mutation of a cell and for quantifying the relatedness of a plurality of genetic mutations. Also presented are systems and apparatuses for performing the subject methods. Further provided are quantitative methods, systems, and apparatuses for selecting information subsets of genes for gene expression analysis. There is no contemplation of the utilization of neonatal or fetal cardiac tissue as a biosynthesis platform for cardioprotective gene expression.

U.S. Pat. No. 6,218,122 provides methods for monitoring disease states in a subject, as well as methods for monitoring the levels of effect of therapies upon a subject having one or more disease states. The methods involve: (i) measuring abundances of cellular constituents in a cell from a subject so that a diagnostic profile is obtained, (ii) measuring abundances of cellular constituents in a cell of one or more analogous subjects so that perturbation response profiles are obtained which correlate to a particular disease or therapy, and (iii) determining the interpolated perturbation response profile or profiles which best fit the diagnostic profile according to some objective measure. In other aspects, the invention also provides a computer system capable of performing the methods of the invention, data bases comprising perturbation response profiles for one or more diseases and/or therapies, and kits for determining levels of disease states and/or therapeutic effects according to the methods of the invention. The patent utilizes the concept of gene profiling to monitor disease states, however there is no conceptual overlap to the instant invention of obtaining a particular genetic response profile related to an age-related response to disease related stressors.

U.S. Pat. No. 6,406,853 is directed toward methods to screen interventions that mimic the effects of calorie restriction. Extensive analysis of genes for which expression is statistically different between control and calorie restricted animals has demonstrated that specific genes are preferentially expressed during calorie restriction. Screening for interventions which produce the same expression profile will provide interventions that increase life span. In a further aspect, it has been discovered that test animals on a calorie restricted diet for a relatively short time have a similar gene expression profile to test animals which have been on a long term calorie restricted diet. The effects of caloric restriction are not relevant to the instant invention.

U.S. Pat. No. 6,468,476 is directed toward bioinformatics methods for enhanced detection of biological response patterns. In one embodiment of the invention, genes are grouped into basis genesets according to the co-regulation of their expression. Expression of individual genes within a geneset is indicated with a single gene expression value for the geneset by a projection process. The expression values of genesets, rather than the expression of individual genes, are then used as the basis for comparison and detection of biological response with greatly enhanced sensitivity. In another embodiment of the invention, biological responses are grouped according to the similarity of their biological profile.

Published Application US20020064788A1 provides methods for identifying new compositions having one or more desired activities, and methods for identifying organisms that are sensitive or resistant to a drug composition. The methods are based upon genetic response profiles generated for an initial set of compositions, where at least one member of the set of compositions has been shown to have at least a first demonstrated activity and a second desired activity. By examining the patterns of genetic and cellular responses (i.e., the genetic response profiles) evoked by a first set of “known” compositions having varying degrees of one or both activities, a preferred pattern of genetic responses can be formulated which corresponds to the desired activity, but not to the demonstrated activity. Additional sets of compounds or compositions can then be screened for the desired genetic response profile, thereby identifying new compositions having the desired activity. Furthermore, populations of organisms can be screened for sensitivity or resistance to drug compositions, based upon comparison of genetic response profiles to the preferred pattern. The reference utilizes genetic profiles to look at responses to drug effects. This approach does not contemplate age-specific effects to identify beneficial targets, as is, the case in the instant invention, nor does it contemplate the use of naturally-occurring, disease related stresses.

Published Application US20030036077 teaches methods for generating an mRNA expression profile from blood. In the subject methods, a population of nucleic acid targets is first generated from an acellular blood sample that contains a plurality of distinct mRNAs, i.e., a disease specific particular blood fraction. The resultant nucleic acid targets are hybridized to an array of nucleic acid probes to obtain an mRNA expression profile. The subject mRNA expression profiles are useful in the identification of disease specific markers. In such applications, the mRNA expression profiles are compared to a control expression profile to identify disease specific markers, where the identified markers subsequently find use in diagnostic applications. The subject methods also find use in diagnostic applications, where the mRNA expression profile is compared to a reference in making a diagnosis of the presence of a disease condition.

WO 00188188A2 provides for examining ischemic conditions, comprising measuring the expression levels of particular genes in a test sample or determining the expression profile of a gene group in the sample comprising a plurality of genes selected from said particular genes and is essentially a method to determine ischemia-inducible genes in tissues. This publication lacks the notion of disease-related stress, and the concept of exploiting the inherently greater protective response in young age exploited by the instant invention.

WO 09923254A1 Measures developmental changes in baseline (i.e; unstressed) gene expression, and thus is conceptually different from the instant invention.

### SUMMARY OF THE INVENTION

The present invention, for the first time, recognizes and advantageously exploits the fact that the capacity to resist stress is greatest during development {i.e; fetal and neonatal stages}, in comparison to the mature counterpart. Based upon this, the present inventors then determine the molecular basis for this difference with the purpose of identifying protective genes which account for this enhanced stress response. The invention is, in large part, predicated upon the concept that the transcript profile represents the “anti-disease network”, rather than causative networks which promote the disease.

Accordingly, it is a primary objective of the instant invention to use age-specific differential gene expression profiling for identifying protective genes which account for enhanced stress response. This was conducted in naturally-occurring disease states and represents a complex pathophysiological stress which is unique, non-artificial and therapeutically relevant.

It is an additional objective of the instant invention to exploit the protective properties of the fetus using gene profiling of the fetal stress response. In this case, the inventors will utilize the instant methodology in fetal hearts from abortuses afflicted with severe forms of congenital heart disease, and thus subject to pathological stress.

It is still a further objective of the instant invention to exploit the enhanced fetal stress response to exogenous (artificial) stimuli designed to provoke a beneficial genetic response, including but not limited, to UV irradiation, environmental toxins, pathogenic organisms, and other noxious stimuli.

It is yet an additional objective of the instant invention to identify a protective gene program which is adapted to hypoxia, and which contains various gene targets revealed by various stresses, which have therapeutic potential in disorders of reduced oxygen supply, such as cerebral vascular disease and ischemic heart disease, by exploiting the fact that, since blood oxygen levels are lower in the fetus during gestation and during and around the time of birth; the fetus, and the newborn to a lesser extent, develop such an innately protective gene program.

It is still an additional objective of the instant invention to define a cardioprotective gene program, simultaneously exhibiting a combination of evident functional clusters including both up-regulated and down-regulated genes evidencing anti-hypertrophic, anti-fibrotic and pro-vasodilatory programs.

It is yet a further objective of the instant invention to provide an early diagnostic tool for determining the presence of cardiac compensation, e.g. by carrying out a process for diagnosing hypertrophic heart disease in a patient comprising obtaining a characteristic differentially expressed cardiac nucleic acid sequence profile from said patient; and comparing said profile to the instantly disclosed cardioprotective gene network.

These and other objectives and advantages of the instant invention will become apparent from the following description wherein are set forth, by way of illustration and example, certain embodiments of this invention.

### BRIEF DESCRIPTION OF THE FIGURES

The patent or application contains at least one drawing executed in color. Copies of this patent or patent application publication with color drawings will be provided by the Office upon request and payment of the necessary fee.

FIG. 1 displays a hierarchical clustering of gene expression data of the 24 patients operated upon for RVOT obstruction;

FIG. 2 illustrates a table of Differentially Expressed Genes, inclusive of their confirmed CloneID and confirmed Unigene Cluster ID.

### DETAILED DESCRIPTION OF THE INVENTION

The above-stated and other related objectives are realized by providing (1) a unique combination of targets which comprise the protective response, in the form of a particular gene expression profile or gene network (inclusive of a combination of genes which may be both up-and down-regulated), constitute, a gene network; and (2) by the recognition of various utilities, either individually, or in particular combinations, which demonstrate enhanced combinatorial effects in the identified gene activation network.

These effects include, but are not limited to:

A) Anti-hypertrophy effects—relating to cardiac muscle cells. Regression of intrinsic cardiac hypertrophy is therapeutically useful.

B) Anti-fibrosis effects—Mitigation of fibrosis in the heart is beneficial.

C) Anti-contractility effects—Down-regulation of intrinsic cardiac contractility is therapeutically useful in certain pathological conditions as a means to reduce energy and/or oxygen demands in the stressed or failing heart.

D) Anti-proliferative effects—relating to vascular smooth muscle cells (VSMC). Prevention of VSMC proliferation would be useful in ameliorating coronary occlusive disorders. More generally, anti-growth properties may be useful as cancer treatment.

E) Wound healing—Identified gene network promotes favorable wound healing and remodeling in response to stress.

F) Cell survival (anti-apoptosis)

With regard to hypertrophic heart disease, most forms of advanced heart disease, regardless of causation, feature hypertrophy of the constituent cardiomyocytes and gross enlargement of the heart. While in the early stages this process is considered beneficial by virtue of increasing cardiac output, however, continued hypertrophic stress eventually leads to decompensated heart failure.

Although the factors that govern this transition from physiological to pathological hypertrophy are not well characterized, treatment directed to limiting the hypertrophic response would be beneficial.

The stressed newborn heart reveals a novel functional module predicted to resist excessive hypertrophy, and it is within the purview of the present invention to utilize the cardioprotective gene program defined therein as an early diagnostic tool for evidencing and characterizing the presence of early, and essentially asymptomatic hypertrophic heart disease.

Thus, the present invention is further directed toward a process for diagnosing hypertrophic heart disease in a patient comprising the steps of (1) obtaining a characteristic differentially expressed cardiac nucleic acid sequence profile from said patient; and (2) comparing said profile to the immature heart cardioprotective gene network as herein set forth. The presence of nucleic acid sequences in said patient's characteristic profile determined to have anti-hypertrophic properties is deemed evidentiary of physiologic compensation of hypertrophic heart disease.

### Methods

Patients

Myocardial samples were taken in 24 patients operated on for obstructive heart lesions, ranging in age from 6 days to 180 months. The samples were acquired immediately after aortic occlusion and stored in liquid nitrogen. The patients were divided into 2 groups. Group I consisted of 7 patients (2 females, 5 males) age ranged from 5 days to 66 days {mean 30 days}; weight ranged from 2.5 kg to 4.9 kg {mean 3.6 kg}. The diagnosis included tetralogy of Fallot (TF) (n=4), complex transposition (n=2), and truncus arteriosus (n=1). Group II consisted of 17 patients (6 females, 11 males) age ranged from 4 months to 180 months {mean 33.5 months}; weight ranged from 5.18 kg to 33.5 kg, {mean 11.3 kg}. The diagnoses in Group II included TF with (n=1) or without (n=16) pulmonary atresia. One patient underwent RV to pulmonary conduit change subsequent to repair of ventricular septal defect and subaortic stenosis by a Damus-Kaye-Stansel procedure.

In accordance with this invention, immature heart tissue is understood to mean myocardial samples taken from patients within the age groupings as set forth above, as well as fetal myocardial tissue.

In accordance with this invention, the terms cardioprotective gene network, cardioprotective gene pattern, cardioprotective gene profile, and cardioprotective gene program are understood to mean a combination of nucleic acid sequences which are up-regulated and down-regulated in neonatal or fetal heart tissue as a result of naturally occurring disease states, e.g. naturally occurring and chronic hemodynamic and/or hypoxic stress, such as that induced by obstructive congenital heart disease.

In accordance with the present invention “Characteristic differentially expressed cardiac nucleic acid sequencing profile” refers to the difference in nucleic acid expression based on analysis of the patient myocardial sample, with direct comparison to normal values determined for a specific laboratory, or in comparison to corresponding data obtained from the same patient at an earlier time point in the clinical course of his disease. Such comparisons are facilitated by the method used in the current invention in which the transcript intensity corresponding to each probe on the array was compared to that corresponding probe in Universal Human RNA Reference sample. Other comparisons which may be informative would include those obtained through in silico database searches consisting of cardiac disease-specific transcriptional profiles.

In accordance with the present invention “Evident Functional Clusters” includes both upregulated and downregulated nucleic acids sequences evidencing cytoprotective, anti-hypertrophic, anti-fibrotic, and other clusters predicted to promote vasodilatation and favorable extra-cellular matrix remodeling and wound healing.

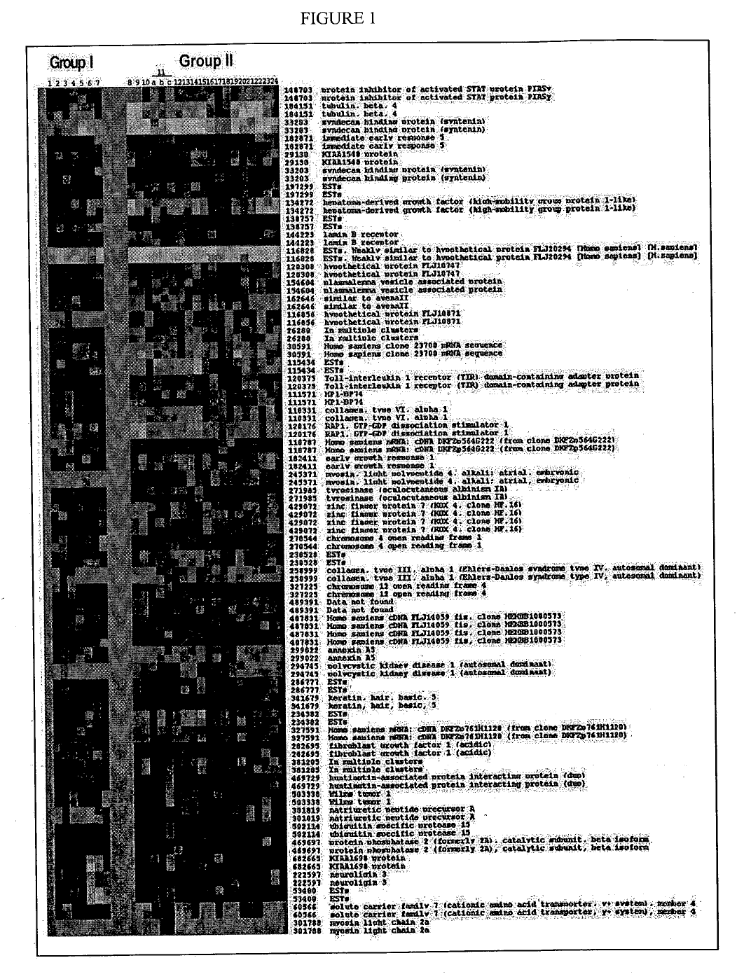

With reference to FIG. 1, the figure displays a hierarchical clustering of gene expression data of the 24 patients operated upon for RVOT obstruction. Each row represents a separate cDNA clone on the microarray and each column an mRNA sample from a separate patient. Patient mRNA samples are separated in 2 groups as indicated on the top. The results presented represent the ratio of hybridization of fluorescent cDNA probes prepared from each patient mRNA sample to a reference mRNA sample, and are a measure of gene-specific expression levels. Red color indicates higher expression, and green color indicates lower expression, relative to the reference sample.

Gene Expression Analysis

RNA Isolation

Total RNA was isolated from tissue samples utilizing TRIZOL reagent according to the protocol outlined by the manufacturer (Gibco/BRL). Briefly, frozen tissues were powdered using a mortar and pestle, cooled in liquid nitrogen then further manually homogenized in a microtube using disposable homogenizers in the presence of the Trizol reagent.

RNA concentrations were determined by spectrophotometric analysis at 260 nm and quality was confirmed by running a 50-250 ng aliquot on the Agilent 2100 Bioanalyzer. All samples were stored at −70° C. until analyzed. Universal Human Reference RNA (Stratagene) was used as the reference sample for all hybridizations.

Arraying Procedure and Processing

Microarrays were manufactured at the University of Toronto Microarray Centre (Toronto, Canada) utilizing cDNAs generated from 19,000 individual cDNAs from Genome Systems (St. Louis, Mo., USA). The CDNA inserts were PCR amplified from the pT7T3D-Pac vectors in 96 well format. Purification of the ESTs was performed using Telechem filter plates (Sunnyvale, Calif., USA) using a Beckman Biomek 2000 robotic workstation. After purification, PCR products were-rearrayed into 384 polypropylene collection plates from Whatmann Polyfiltronics Inc. (Rockland, Mass., USA). The amplified purified cDNAs were spotted using the SDDC-2 robotic arrayer from Virtec Engineering Services Incorporated (VESI, Toronto, Canada). The cDNAs were arrayed using 32 Stealth Chipmaker 3 Microspotting pins from Telechem International (Sunnyvale, Calif.) onto Corning CMT-GAPS™ slides (Corning, N.Y., USA). Each of the 32 pins prepared a 25 row, 24 column grid. The resultant pattern is 32 grids, in an 8×4 pattern, each with 600 spots. Each individual spot measures approximately 120 μm in diameter. The spots were printed at a centre-to-centre distance of 170 μm.

Names and identifications of all nucleic acid sequences, inclusive of EST's which are part of the instantly disclosed cardioprotective gene network were deduced via the UNIGENE data bank, and each of the individual UNIGENE Cluster ID reports and all related citations and sequence listings associated therewith are herein incorporated by reference, as if they were a part of the original specification.

Fluorescence Labeling of cDNA

Total RNA (10 μg) from either the patient or reference sample was added to a reaction mixture containing the following: 8 μl 5× first strand buffer (Invitrogen, Burlington, Canada), 1.5 μl AncT primer (T₂₀VN, Cortec, Kingston, Canada), 3 μl of a 7 mM dNTP mix (final concentration of 500 μM dATP, aTTP and dGTP each), 50 μM dCTP, 25 μM Cyanine3-dCTP or Cyanine5-dCTP (PE/NEN, USA), 10 mM DTT. Final reaction volumes were brought up to 40 μl with water and primer annealing was initiated by heating the reaction mix to 65° C. for 5 minutes then 42° C. for 5 minutes. Reactions were initiated by the addition of 2 μl of Superscript II RT (200 units/μl, Invitrogen, Burlington, Canada) and allowed to proceed for 2 hours at 42° C. Reactions were then terminated by the addition of 5 μl of 50 mM EDTA (pH8.0). RNA was degraded with the addition of 2 μl of 10 N NaOH and heating to 65° C. for 20 minutes. After RNA degradation, samples were neutralized by adding 5 μl of 5 M acetic acid to the reaction volume. Samples were then combined (patient and reference pairs) and added to 400 μl of sterile water followed by application to PCR spin columns (Millipore, Canada). Samples were centrifuged at 1000×g for 15 minutes at room temperature. Labeled sample was recovered by adding 5 μl of water to the membrane, inverted and spun at 1000×g for 2 minutes. Samples were then used immediately for hybridization.

Hybridization

Resuspended samples were added to a hybridization mixture containing 80 μl DIG EASYHYB (Roche, Mississauga, Canada), 4 μl yeast tRNA (10 μg/μl), and 4 μl salmon sperm DNA (10 μg/μl, Sigma, Mississauga, Canada). Samples were heated to 65° C. for 2 minutes, cooled briefly and centrifuged to bring down any condensate that may have accumulated during heating. The entire volume was applied to the microarray and placed in a sealed, humidified hybridization chamber and incubated over night at 37° C. Slides were then washed consecutively in 1×SSC, 0.1% SDS for 3×10 minutes at 50° C. A final rinse was carried out at room temperature in 0.1×SSC for 5 minutes and the slides were then centrifuged for 5 minutes at 500 rpm to dry.

Scanning and Quantification

Slides were scanned on a scanning laser fluorescence confocal microscope (ScanArray 4000XL, Perkin Elmer, Mass., USA). Individual 16-bit TIFF images were obtained by scanning for each of the two fluors. An overlay image of the two images was created and quantified utilizing the QuantArray (v2.1) program (Perkin Elmer, Mass., USA). Intensity values for each spot were normalized and ratios calculated resulting in a value of patient sample/reference. Individual spots had to pass a number of quality criteria to be included in the data analysis, including a minimum spot/local background intensity ≧1, a minimum spot/mean background intensity ≧1, and a minimum spot intensity of 100.

Data Analysis

Data were stored in and analyzed with the GeneTraffic Microarray Database and Analysis System (Iobion Informatics, La Jolla, USA), as well as the Significance Analysis for Microarrays (SAM) program. Scanned 16-bit TIFF images representing each hybridized microarray slide and the associated quantification data files were entered into the database with a complete annotation of the experiments based on the current MIAME standards for microarray experiments.

Each hybridization data set was normalized using Lowess subarray normalization. Lowess normalization uses a local weighted smoother to generate an intensity dependent normalization function. In subarray normalization each subarray or grid is normalized individually to correct for variation in local mean signal intensities across the surface of the arrayⁱⁱ. The resultant normalized log2 patient/sample intensity ratios were used for statistical analysis. A repeated permutation procedure was performed to ascertain potential statistical differences in gene expression between the two age groupsⁱ. The median false discovery rate, based on analysis of permuted data sets, was less than 1.0% and only genes with a minimum 2 fold change in expression were selected. Results from the SAM analysis were visualized as hierarchical clusters.

Validation using QPCR

Independent confirmation of increased transcription levels was performed on 4 randomly selected genes showing increased neonatal expression using real-time quantitative polymerase chain reaction (qPCR). Primers were constructed against the 3′ ends of fibroblastic growth factor 1 (acidic), HDGF, syntenin, and egr-1 and amplicon abundance determined in real-time by SYBR Green Dye (Applied Biosystems) fluorescence measurement during the logarithmic phase and normalized to that of a control gene, cyclophilin. Fold changes for the cyclophilin-normalized value of each transcript were determined as a ratio of sample patient to that of the Universal Human Reference RNA. Multiple regression analysis was performed to compare intergroup differences in transcript fold changes determined by microarray analysis versus qPCR for each of the selected genes.

Results

All patients survived the surgical procedure and were discharged from hospital. One neonate with Taussig-Bing anomaly plus atrioventricular septal defect required postoperative extra-corporeal membrane oxygenation. There were no significant differences in pre-operative arterial saturation between the two age groups (Group I: 79.85+ 12.5 vs Group II: 87.24+ 12.9; p=0.21). There were no differences in preoperative central venous pressure (Group I: 7.2+ 2.3 vs Group II: 7.4+ 2.6; p=0.85) or postoperative inotropic support (dopamine: Group I mean 4.5+ 2.74 vs 4.5+3.01; p=1.0; milrinone: group I: 0.53+ 0.29 vs Group II: 0.26+ 0.31; p=0.11).

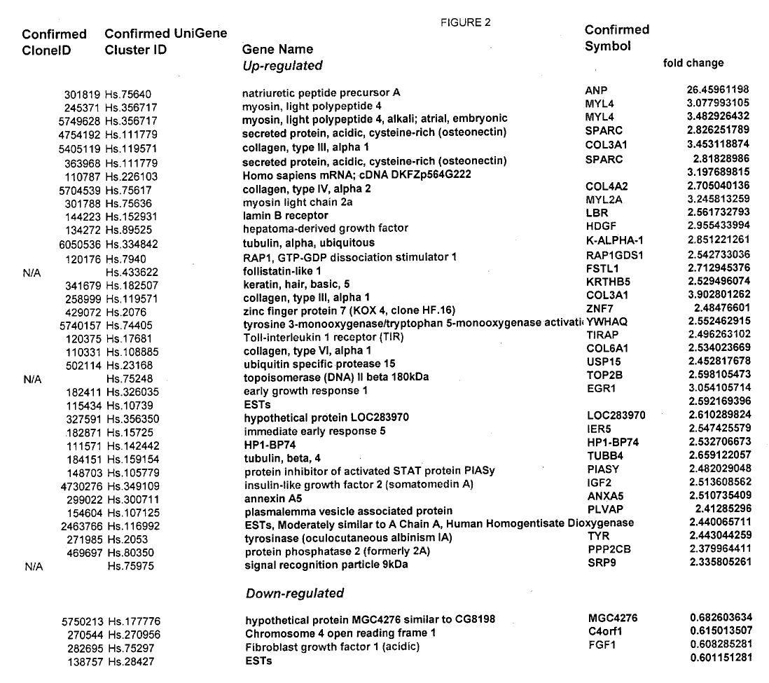

In accordance with FIG. 2, a table of Differentially Expressed Genes is shown. Genes with higher expression levels in Group I (neonatal) are indicated by fold change >1; genes with lower expression levels in Group I (neonatal) are indicated by fold change <1. Red typeface, literature-validated cardioprotective gene elements; blue typeface, gene elements with predicted anti-hypertrophic properties; black typeface, gene elements with unprecedented or unanticipated cardiovascular effects.

Note that with reference to FIG. 2,for a confirmed clone ID indicated “N/A”, this means the clone used links to a confirmed Unigene cluster ID; the sequence similarity among clone IDs does not permit identification of the specific clone ID which links to the Unigene cluster ID.

Significant genes were searched using The Stanford Online Universal Resource for Clones and ESTs (SOURCE), which compiles information from several publicly accessible databases, including UniGene, dbEST, Swiss-Prot, GeneMap99, RHdb, GeneCards and LocusLink.

Several genes are literature-validated {Pub Med January 2003} as having a protective effect against experimentally-induced myocardial ischemic/reperfusion injury, including atrial natriuretic polypeptide (ANP), myosin light chains 4 (MYL4) and 2a (MYL2a), hepatoma-derived growth factor (HDGF), and toll-interleukin 1 receptor (TIR) (highlighted red in FIG. 2). Several additional genes have documented anti-growth properties and may be speculated to resist cardiac hypertrophy and promote vasodilatation, including the small GTPase rap 1, the transcriptional repressor zinc finger protein 7, protein phosphatase 2 (PPP2A), ubiquitin specific protease 15, and egr-1. The finding of decreased transcript expression of fibroblastic growth factor 1 (acidic) (highlighted blue in FIG. 2} would be predicted to confer additional net anti-proliferative effects. Several genes have been previously designated as “fetal” genes, including ANP, HDGF, and keratin, hair, basic, 5. The remaining genes are unprecedented in terms of predicted cardiovascular effects (black typeface in FIG. 2).

Targets/Subset Profiles of Particular Interest

Atrial Natriuretic Polypeptide (ANP) The effects of ANP are mediated through binding to the A-type natriuretic peptide receptor which activates guanyl cyclase, leading to the formation of cGMP^{iii,iv}. Upregulation of ANP expression occurs in all four cardiac chambers in response to acute and chronic hypoxic stress^{v,vi,vii}, implying that the ANP may represent an hypoxia-inducible gene per se, the regulation of which can occur independently of changes in pulmonary artery pressure and ventricular hypertrophy. The fact that there were no significant differences in saturation levels between the 2 age groups argues that the increased ANP response observed neonatally reflects an age-dependent enhancement to hypoxic signaling rather than a response commensurate with a greater degree of hypoxia. Similarly, the lack of intergroup differences in CVP rules out stretch-induced ANP activation as an explanation for differential expression.

The direct effect of ANP on myocardial ischemia/reperfusion injury is unknown; however, the fact that ANP elevates cGMP levels, inhibits pro-apoptotic p38 MAPK activation, and antagonizes tumor necrosis factor-α-induced changes in endothelial cell cytoskeleton and prevents macromolecule permeability changes^{viii}, is highly suggestive of a novel cytoprotective effect in this context. Recombinant ANP peptide has been shown to potentiate myocardial ischemic preconditioning through a nitric oxide-dependent mechanism^{ix}. Additional cytoprotective effects may accrue from upregulation of toll-interleukin 1 receptor^{,x}, attributable to activation of ischemic preconditioning and anti-apoptotic signaling pathways, respectively.

It is unknown as to whether ANP gene induction in the heart confers a direct cytoprotective effect against excessive, or pathological, hypertrophic or hypoxic stimuli, independently of its vasoreactive and natriuretic properties. Consistent with this prediction, however, is the observation that exogenous or endogenous ANP peptide suppressively regulates the cardiac hypertrophic response in an autocrine/parcrine manner by increasing myocyte cGMP levels in neonatal rat cardiomyocytes in vitro^{xii}, and that transgenic mice over expressing ANP have lower heart weights under normoxic conditions and an attenuated right ventricular hypertrophic response to hypoxia-induced pulmonary hypertension^{xiii}.

A decline in ventricular ANP gene transcription normally occurs postnatally concurrently with a switch from right to a left ventricular dominant circulation^{xiv,xv,xvi}, providing an explanation for neonatal expression of this transcript in the presence of RVOT obstruction. However, the fact that expression levels of two other hypertrophy-associated genes, gene β-myosin heavy chain and endothelin-1, were not found to be differentially expressed in our study, argues that neonatal upregulation of ANP is functionally important and not simply a marker of hypertrophic stress. While not wishing to be bound to any particular theory, we speculate that ANP gene activation in the context of neonatal obstructive heart disease may serve to mitigate excessive hypertrophic signaling and protect against the transition from physiological to pathological hypertrophy.

Protein phosphatase 2A (PP2A) Transgenic mice over-expressing protein phosphatase 2A exhibit reduced cardiac contractility and progressive ventricular dilatation, an effect which may serve to mitigate the concentric hypertrophic response inherent in neonatal TFxvii, and which may be attributable to PP2A-mediated antagonism to calcium calmodulin-dependent protein kinase activity^{xviii}. In vascular smooth muscle cells PP2A inhibits platelet-derived growth factor BB-mediated phosphorylation of BAD and forkhead transcription factor FKHR-L1, and this effect correlates with increased apoptosis^{xix}. The anti-hypertrophic signaling described for this phosphatase may thus be predicted to complement favorable cardiac vascular remodeling attributable to increased ANP.

Early growth response 1 (egr-1) is a zinc finger transcription factor which exerts opposing effects depending on the latency of the measured response and the contextual pattern of co-regulated gene expression. For example, growth factors and cytokines including platelet-derived growth factor, angiotensin II, tumor necrosis factor-α (TNF-α) and interleukin-1β increase egr-1 message within 15 minutes^{xx}, which, in turn, activates transcription of several genes implicated in the pathogenesis of vascular diseases, including TNF-α^{xxi}, PGDF^{xxii}, interleukin-2^{xxi}, and fibroblastic growth factor (FGF)^{xxiii}, producing an positive amplification loop favoring smooth muscle cell proliferation.

Conversely, egr-1 exerts a counter-regulatory effect through a sustainable transactivation of peroxisome proliferator-activated receptor γ1 (PPAR γ1), itself a ligand-activated nuclear transcription factor which potently suppresses growth factor- and cytokine-mediated signaling in vascular smooth muscle^{xxiv,xx}, possibly accounting for the reduced FGF message observed in the neonatal group. Thus, in addition to mitigation of hypertrophic cardiomyocyte signaling, coordinated expression of ANP, egr-1 and protein phosphatase 2A would be predicted to favor vascular smooth muscle regression promoting coronary vasodilatation and having the effect of augmenting oxygen delivery to hypertrophic, hypoxically perfused myocardium. Although not literature-validated as cardiac ‘targets’, increased neonatal expression of the small GTPase rap 1, which inhibits the extracellular signal-related kinase (ERK) signaling cascade^{xxv}, and the transcriptional repressor, zinc finger protein 7^{xxvi}, could plausibly further limit hypertrophic responses in the hemodynamically stressed heart.

Several genes differentially expressed in the neonatal group conceivably augment the capacity for matrix remodeling and cellular regeneration, including the re-expression, or more likely, persistence, of ‘fetal’ genes, implying parallels between physiological fetal and stress-induced tissue remodeling. HGDF is a nuclear-targeted growth factor conspicuously expressed in embryonic ventricular myocytes, endocardium, and cells of the ventricular outflow tract, implying a role in cardiovascular growth and differentiation^{xxviii}. Although not specified as a fetal gene, egr-1 also has wound healing properties by virtue of capacity to stimulate angiogenesis^{xxix} and endothelial production of membrane type 1 matrix metalloproteinase^{xxx}. Egr-1-mediated upregulation of plasminogen activator inhibitor-1 (also known as serpine-1)^{xxxi} may serve an important adaptive function, increasing the fibrin stroma on which neoangiogenesis and tissue repair may take place^{xxxii}.

An unexpected finding of global gene analysis of heart tissue in this study was the evidence for anti-growth properties of several transcripts, including tumor suppressor genes egr-1, ubiquitin specific protease 15, the transcriptional repressor zinc finger protein 7, in concert with reduced levels of FGF 1 mRNA. This anti-growth program is thematically consistent with a greater compensatory reaction to hyperproliferative signals in the immature heart, which may serve to protect against the development of pathological interstitial fibrosis.

Intergroup differences in 4 randomly selected transcripts levels determined by microarray analysis were highly correlated with those determined by qPCR: multiple R² value=0.998; p=0.001.

Neither the cardioprotective gene expression profile, per se, nor the subsets identified as having particular utilities have been heretofore recognized or suggested.

### CONCLUSIONS

Of the estimated 32,000 to 38,000 genes encoded by the human genome, approximately 20,000 to 25,000 are thought to be expressed in the cardiovascular system. The combinatorial pattern of gene expression in the heart serves to increase the repertoire of responses to pathological stress, and it is intuitively logical that the capacity for such adaptive responses is inversely related to the fetal-neonatal-adult development gradient. The results of the current microarray-based gene expression profiling study confirm the existence of a protective re-programming response which is most evident in neonatal myocardium subject to the hemodynamic and metabolic stress imposed by structural congenital heart disease.

Within the overall disease-specific expression profiles, additional subsets have also been implicated which define novel molecular targets in the pathogenesis of human cardiovascular disease, including cardiac hypertrophy^{xxxiii}, dilated cardiomyopathy (DCM)^{xxxiv}, and the clinical response to. β-blockade in patients with DCM^{xxxv}.

The molecular signatures identified using this approach are typically construed as being either mechanistically relevant to the disease pathogenesis, or alternatively, as markers of disease progression. In contrast, it has been determined by the present inventors, that this approach can be used to identify endogenous patterns of gene expression which are activated in response to the primary disease-causing pathway, and have the effect of generating a counteracting, and highly adaptive pattern of gene activation, which serve to suppress aberrant disease-related molecular pathways.

The age-related differential transcript profile observed in our study included upregulation of the majority (42/50) of significant genes, consistent with the idea of a more robust response in the neonatal heart. The literature-validated function of the genes implicated in this response suggested a clustering of overlapping ‘themes’ or sub-profiles relating to cytoprotection, anti-hypertrophic remodeling, and re-expression of fetal genes.

All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures. One skilled in the art will readily appreciate that the present invention is well adapted to carry out the objectives and obtain the ends and advantages mentioned, as well as those-inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

Files Referenced In Specification

ⁱTusher V G, Tibshiran R, Chu G. Significance analysis of microarrays applied to the ionizing radiation response. PNAS 2001;5116-5121.

ⁱⁱHolloway A J, vanLaar R K, Tothill R W, Bowtell D D L. Options available from start to finish for obtaining data from DNA microarraysII. Nature Genetics 2002;32:481-489.

ⁱⁱⁱBrunner-La Rocca H P, Kiowski W, Ramsay D, Sutsch G. Therapeutic benefits of increasing natriuretic peptide levels. Cardiovascular Research 2001;51:510-520.

^{iv}Boomsma F, van den Meiracker A H. Plasma A- and B-type natriuretic peptides: physiology, methodology and clinical use. Cardiovascular Research 2001;51:442-449.

^{v}Chen Y-F, Durand J, Claycomb W C. Hypoxia stimulates atrial natriuretic peptide gene expression in cultured atrial cardiocytes. Hypertension 1997;29:75-82.

^{vi}Chen Y F, Elton T S, Bounelis P, Jin H, Oparil S. Acute hypoxia increases atrial natriuretic peptide (ANP) gene expression in left atrium of the rat. Clin Res 1993;38:18.

^{vii}Chen Y F, Elton T S, Bounelis P, Jin H, Oparil S. Altered atrial natriuretic peptide gene expression in atria of rats adapted to chronic normobaric hypoxia. Am J Hypertension 1989;2:45.

^{vii}Kiemer A K, Weber N C, Furst R, Bildner N, Kulhanek-Heinze S, Vollmar A M. Inhibition of p38 MAPK activation via induction of MKP-1 atrial natriuretic peptide reduces TNF-α-induced actin polymerization and endothelial permeability. Circulation Research 2002;90:874-881.

^{ix}Hirohisa O, Hitoshi H, Shigetoshi M, Yuklya N, Masataka Y, Kenichi N, Kenji K, Kunio A, Keiichiro K, Shinjiro S. Alpha-human atrial natriuretic peptide mimics ischemic preconditioning through NO-PKC-mitochondrial KATP channel dependent mechanism. Circulation 2002;106;11-345.

^{x}Vabulas R M, Ahmad-Nejad P, Ghose S, Kirschning C J, Issels R D, Wagner H. HSP70 as endogenous stimulus of the toll/interleukin-1 receptor signal pathway. J Biol Chem 2002;277:15107-12.

^{xi}Saito A, Hayashi T, Okuno S, Ferrand-Drake M, Chan P H. Overexpression of copper/zinc superoxide dismutase in transgenic mice protects against neuronal cell death after transient focal ischemia by blocking activation of the Bad cell death signaling pathway. Journal of Neuroscience 2003;23(5):1710-8.

^{xii}Horio T, Nishikimi T, Yoshihara F, Matsuo H, Takishita S, Kangawa K. Inhibitory regulation of hypertrophy by endogenous atrial natriuretic peptide in cultured cardiac myocytes. Hypertension 2000;35:19-24.

^{xiii}Klinger J R, Petit R D, Curtin L A, Warburton R R, Wrenn D S, Steinhelper M E, Field L J, Hill N S. Cardiopulmonary responses to chronic hypoxia in transgenic mice that overexpress ANP. J Appl Physiol 1993;75:198-205.

^{xiv}Cantin M, Gutkowska J, Thibault G, Milne RW, Ledoux S, MinLi S, Capeau C, Garcia R, Hamet P, Genest J. Immunocytochemical localization of atrial natriuretic factor in the heart and salivary glands. Histochemistry 1984;80:113-127.

^{xv}Nemer M, Lavigne J P, Drouin J, Thibault G, Gannon M, Antakly T. Expression of atrial natriuretic factor gene in heart ventricular tissue. Peptides 1986;6: 1147-1152.

^{xvi}Kim S H, Han J H, Cao C, Kim S Z, Cho K W. Postnatal changes in inhibitory effect of C-type natriutetic peptide on secretion of ANP. Am J Physiol Regulatory Integrative Comp Physiol 2002;282:R1672-R1679.

^{xvii}Gergs U, Fabritz L, Matus M, Boknik P, Schmitz W, Neumann J. Impaired ventricular contractility and cardiac hypertrophy in transgenic mice overexpressing the protein phosphatase 2A in the heart. Circulation 2002; 106:II-99.

^{xviii}Westphal R S, Anderson K A, Means A R, Wadzinski B E. A signaling complex of Ca2+-calmodulin-dependent protein kinase IV and protein phosphatase 2A. Science 1998;280:1258-61.

^{xix}Yellaturu CR, Bhanoori M, Neeli I, Rao GN. N-Ethylmaleimide inhibits platelet-derived growth factor BB-stimulated Akt phosphorylation via activation of protein phosphatase 2A. J Biol Chem 2002;277:40148-40155.

^{xx}Fu M, Zhang J, Lin Y, Zhu X, Ehrengruber M U, Chen Y E. Early growth response factor-1 is a critical transcriptional mediator of peroxisome proliferator-activated receptor-γ1 gene expression in human aortic smooth Muscle cells. J Biol Chem 2002;227:26808-26814.

^{xxi}Takano H, Nagai T, Asakawa M, Toyozaki T, Oka T, Komuro I, Saito T, Masua Y. Peroxisome proliferator-activated receptor activators inhibit lipopolysaccharide-induced tumor necrosis factor-α expression in neonatal rat cardiac myocytes. Circ Res 2000;87:596-602.

^{xxii}Khachigian L M, Linder V, Williams AJ, Collins T. Egr-1-induced endothelial gene expression: a common theme in vascular injury. Science 1996;271:1427-1431.

^{xxiii}Biesiada E, Razandi M, Levin E. Egr-1 activates basic fibroblast growth factor transcription J Biol Chem 1996;271:18576-18581.

^{xxiv}Inoue M, Itoh H, Tanaka T, Chun T H, Doi K, Fukunaga Y, Sawada N, Yamshita J, Masatsugu K, Saito T, Sakaguchi S, Sone M, Yamahara K, Yrugi T, Nakao K. Oxidized LDL regulates vascular endothelial growth factor expression in human macrophages and endothelial cells through activation of peroxisome proliferator-activated receptor-gamma. Arterioscler Thromb Vasc Biol 2001;21:560-566.

^{xxv}Stork P J, Schmitt J M. Crosstalk between cAMP and MAP kinase signaling in the regulation of cell proliferation. Trends Cell Biol 2002; 12(6):258-266.

^{xxvi}Feduchi E, Gallego M I, Lazo P A. The human zinc-finger protein-7 gene is located 90 kb 3′ of MYC and is not expressed in Burkitt lymphoma cell lines. Int J Cancer 1994;58(6):855-859.

^{xxvii}Couchman J R, Chen L, Woods A. Syndecan and cell adhesion. Int Rev Cytol 2001;207:113-50.

^{xxviii}Everett A D. Identification, cloning, and developmental expression of hepatoma-derived growth factor in the developing rat heart. Dev Dyn 2001;222(3):450-458.

^{xxix}Abe M, Sato Y. cDNA microarray analysis of the gene expression profile of VEGF-activated human umbilical vein endothelial cells. Angiogenesis 2001;4(4):289-298.

^{xxx}Yamaguchi S, Yamaguchi M, Yatsuyanagi E, Yun S S, Nakajima N, Madri J A, Sumpio B E. Cyclic strain stimulates early growth response gene product 1-mediated expression of membrane type 1 matrix metalloproteinase in endothelium. Lab Invest 2002;82(7):949-956.

^{xxxi}Yan S F, Fujita T, Lu J. Egr-1, a master switch coordinating upregulation of divergent gene families underlying ischemic stress. Nat Med 2000;6:1355-1361.

^{xxxii}Ten V S, Pinsky D J. Endothelial response to hypoxia: physiologic adaptation and pathologic dysfunction. Curr Opin Crit Care 2002;3:242-250.

^{xxxiii}Friddle C J, Koga T, Rubin E M, Bristow J. Expression profiling reveals distinct sets of genes altered during induction and regression of cardiac hypertrophy. Proc Natl Acad Sci USA 2000;97:6745-6750.

^{xxxiv}Hwang J-J, Allen P D, Tsent G C, Lam C-W, Fananapazir L, Dzau V J, Liew C-C. Microarray gene expression profiles in dilated and hypertrophic cardiomyopathic end-stage heart failure. Physiological Genomics 2002;10:31-44.

^{xxxv}Lowes B D, Gilbert E M, Abraham W T, Minobe W A, Larrabee P, Ferguson D, Wolfel E E, Lindenfeld J, Tsvetkova T, Robertson A D, Quaife R A, Bristow M R. Myocardial gene expression in dilated cardiomyopathy treated with beta-blocking agents. N Engl J Med 2002;346:1357-1365.

What is claimed is:

1. A cardioprotective gene network which consists essentially of a combination of nucleic acid sequences which are up-regulated and down-regulated in immature heart tissue as a result of naturally occurring disease states:
wherein said up-regulated nucleic acid sequences consist of Hs.75640, Hs.356717, Hs.356717, Hs.111779, Hs.119571, Hs.111779, Hs.226103, Hs.75617, Hs.75636, Hs.152931, Hs.89525,Hs.334842,Hs.7940,Hs.433622, Hs.182507, Hs.119571, Hs.2076, Hs.74405, Hs.17681, Hs.108885, Hs.23168, Hs.75248, Hs.326035, Hs.10739, Hs.356350, Hs.15725, Hs.142442, Hs.159154, Hs.105779, Hs.349109, Hs.300711, Hs.107125, Hs.116992, Hs.2053, Hs.80350, Hs.75975; and wherein said down-regulated nucleic acid sequences consist of Hs.177776, Hs.270956, Hs.75297, and Hs.28427.

2. The cardioprotective gene network of claim 2, wherein said naturally occurring disease state is obstructive congenital heart disease.

3. A process for identification of a cardioprotective gene program in the immature heart comprising:
providing a sample of immature heart tissue which has been subjected to naturally occurring, chronic hemodynamic and/or hypoxic stress;
carrying out differential gene expression profiling on said sample; and
determining a pattern of up-regulated and down-regulated nucleic acid sequences therein;
whereby a differentially expressed profile of nucleic acid sequences constituting a cardioprotective gene program is identified.

4. The process of claim 3, wherein said naturally occurring, chronic hemodynamic and/or hypoxic stress is associated with obstructive congenital heart disease.

5. The process of claim 3, wherein said cardioprotective gene program simultaneously exhibits a combination of evident functional clusters including both up-regulated and down-regulated nucleic acid sequences evidencing anti-hypertrophic, anti-fibrotic and pro-vasodilatory programs.

6. A process for diagnosing hypertrophic heart disease in a patient comprising:
obtaining a characteristic differentially expressed cardiac nucleic acid sequence profile from said patient; and
comparing said profile to said cardioprotective gene network defined in claim 1;
whereby hypertrophic heart disease is diagnosed.
